(19) United States
(12) Patent Application Publication
(10) Pub. No.: US 2008/0076171 A1
(43) Pub. Date: Mar. 27, 2008

(54) Air germ collector

(75) Inventors: Hans-Dieter Hempel, Mainaschaff (DE); Volker-Joachim Friemert, Kirchheim (DE)

Correspondence Address: COLLARD & ROE, P.C., 1077 NORTHERN BOULEVARD, ROSLYN, NY, 11576, US

(73) Assignee: Biotest AG

(21) Appl. No.: 11/903,057

(22) Filed: Sep. 20, 2007

(30) Foreign Application Priority Data
Sep. 22, 2006 (DE) 10 2006 044 546.5

(51) Int. Cl. C12M 1/00

(52) U.S. Cl. 435/307.1

(57) ABSTRACT

An air germ collector has a defined relative movement between collection medium and a screen cover. The accommodation, with the collection strip secured therein, can rotate, and the screen cover is fixed in place. Likewise, the screen cover can be structured so that it can rotate, if the accommodation is fixed in place. The collection strip can be moved in the accommodation, in order to obtain the required relative movement. The germ-charged air is transported in the following manner: Through the central opening of the cover and passage openings, the air reaches the surface of the collection medium in depressions of the collection strip, where the inertial deposition of the airborne germs takes place. The air continues to flow past the accommodation into the outer drum space, and is drawn through the oblong holes by the fan, and ejected at the lower end of the air germ collector.

### BACKGROUND OF THE INVENTION

1. Field of the Invention

The present invention relates to air germ collectors, particularly air germ collectors having an accommodation for accommodating a collection medium and acceleration means for accelerating air charged with germs, in order to allow inertial deposition of the germs from the air onto the collection medium.

2. The Prior Art

Air germ collectors are used for checking or detecting the air purity in laboratories, hospitals, production clean rooms, etc.

Air germ collectors bring air charged with germs into contact with a collection medium, often a sterile agar medium, so that germs from the air deposit onto the medium. By incubation, the number of germs deposited can be determined using the number of colonies that form. As a rule, the air to be examined is brought into a controlled flow over or through the medium, in order to determine the number of germs per air volume unit.

In addition to filter methods, in which the air to be examined is drawn through a filter, and the so-called impinger method, in which the air to be sampled is passed through a fluid, impaction methods have particularly become established.

Filter methods achieve the deposition of all the germs that are larger than the size of the filter pores, but germs capable of reproducing tend to dry out on the filter. Furthermore, handling is complicated, since the filter media (formerly often paper, today mostly gelatin) must first be placed into cassettes under sterile conditions, removed from the cassettes after sampling, and finally dissolved and applied to agar plates. The impinger method avoids drying out, but because of the handling of fluids, it is also complicated. Furthermore, it frequently leads to different results than comparable methods, because agglutinates can dissolve in the fluid, and the individual germs that are counted as a result lead to a higher colony count.

With the aforementioned impaction methods, inertial deposition (also known as inertial interception) of the airborne germs takes place in a baffle flow or in a centrifugal field. In the case of simple methods, which can be implemented very inexpensively, an agar plate is disposed below or behind a screen cover (screen plate), through which the germ-charged air flows. In the baffle flow, the germs drop onto the nutrient medium, while the air flows away to the side. Since the air always impacts the agar at defined locations, namely below the screen holes (also called gas passage openings in the following), dent-like regions can occur at these locations due to drying out. Furthermore, the germ count determined tends to be too low, since germs always impact the same locations below the screen holes, and therefore several colony-forming units can be counted as one. Also, shear forces that occur during passage through the screen holes can weaken the reproductivity of germs.

In the so-called Hycon method, the inertial deposition of the airborne germs takes place in a centrifugal field. Air that is drawn in is accelerated by rotor blades, and impacts a centrifuge surface provided with agar, which also rotates. This is a method that is not gentle on the germs, but as a rule, obtains reliable results, because the system is closed and easy to validate. In this connection, the agar is situated in depressions of a collection strip that is inserted into the centrifuge before the collection process, and removed afterwards. Sometimes, eccentricities can occur because the strip rotates.

### SUMMARY OF THE INVENTION

It is therefore an object of the invention to provide an air germ collector that substantially eliminates the aforementioned disadvantages. It is another object to provide an air germ collector that is easily built as a compact and portable device, and offers great reliability and counting accuracy, particularly even in the case of varying throughput.

This task is accomplished by means of an air germ collector having
an accommodation for accommodating a collection medium,
acceleration means for accelerating air charged with germs, in order to allow inertial deposition of the germs from the air onto the collection medium, and
a cover for the collection medium, provided with passage openings for the accelerated air.

The cover and the collection medium are movable relative to one another in a defined manner during the inertial deposition. The air germ collector can be designed for the purpose of performing a relative movement between the cover and collection medium that is controlled over time, so that a predetermined relative path and/or angle movement takes place during a predetermined time of the collection process.

The collection medium can be contained in a collection strip interchangeably held in the accommodation, and the predetermined relative path or angle movement during the predetermined time corresponds to the useful length of the collection strip provided with collection medium.

In one embodiment, the passage openings are disposed in a repeating pattern, and the air germ collector is designed for the purpose of performing a relative movement between the cover and the collection medium, during the predetermined time of the collection process. This movement corresponds to the distance or a whole-number multiple of the distance at which the pattern repeats.

In another embodiment, the accommodation is drum-shaped, and the cover is structured as a perforated drum disposed coaxial to the accommodation, in the drum interior surrounded by the accommodation. The passage openings are uniformly distributed over the circumference of the drum mantle. The accommodation is structured so that it can rotate, and the perforated drum is structured to be fixed in place.

The acceleration means can comprise a fan disposed coaxial with the accommodation. The fan can be disposed outside of the drum interior surrounded by the accommodation.

In another embodiment, the collection medium is contained in a collection strip interchangeably held in the accommodation. The collection strip is structured to be movable relative to the accommodation. The air germ collector can have intake means for drawing the collection strip into the accommodation in an automated manner. A common drive can be provided for the intake means and for moving the collection strip relative to the accommodation during the collection process.

In one embodiment, the passage openings are round and/or are uniform in shape, relative to one another. Preferably, the distance between adjacent passage openings, relative to one another, measured from the edge of each of the passage openings, is greater than the diameter of a circle inscribed in a passage opening. More preferably, distance between adjacent passage openings relative to one another, measured from the edge of each of the passage openings, is less than five times the diameter of a circle circumscribed around a passage opening.

The passage openings preferably have a minimum area of 0.2 square millimeters each, and a maximum area of 1 square millimeter each.

In one embodiment, the cover has bypass openings that have an area greater than the area of a passage opening. Preferably, the bypass openings can be closed.

The air germ collector according to the invention can be configured as a portable device and can also have means for detecting data that characterize the collection process. Furthermore, the device can have means for wireless transmission of the data that characterize the collection process to an external receiver.

Fundamentally, any variant of the invention described or indicated within the scope of the present application can be particularly advantageous, depending on the economic and technical conditions in an individual case. Unless something is said to the contrary, or to the extent that it can fundamentally be implemented technically, individual characteristics of the embodiments described are interchangeable or can be combined with one another and also with measures known from the state of the art.

### BRIEF DESCRIPTION OF THE DRAWINGS

In the following, examples of preferred embodiments of the present invention will be explained in greater detail, using the related drawings. In this connection, the drawings are purely schematic, and are not representations to scale, for reasons of a clear illustration. In particular, ratios of the dimensions can deviate from actual embodiments.

Elements that correspond to one another are provided with the same reference symbols in the individual figures.

In the drawings:

FIG. 1 shows a perspective exploded view of a particularly preferred embodiment of an air germ collector according to the invention, whereby the lower housing part is shown broken off and the cover hood is shown in section;

FIG. 2 shows a half-sided cross-section of the collection part of an air germ collector structured similar to FIG. 1;

FIG. 3a-b show, in stylized form, the drum-shaped accommodation as well as a screen drum insert of an air germ collector according to the invention, in a top view, as well as different stages of drawing in a collection strip structured in accordance with FIG. 3c; and

FIG. 3c shows a simple embodiment of a collection strip structured for use with an air germ collector according to the invention, in a top view.

### DETAILED DESCRIPTION OF THE PREFERRED EMBODIMENT

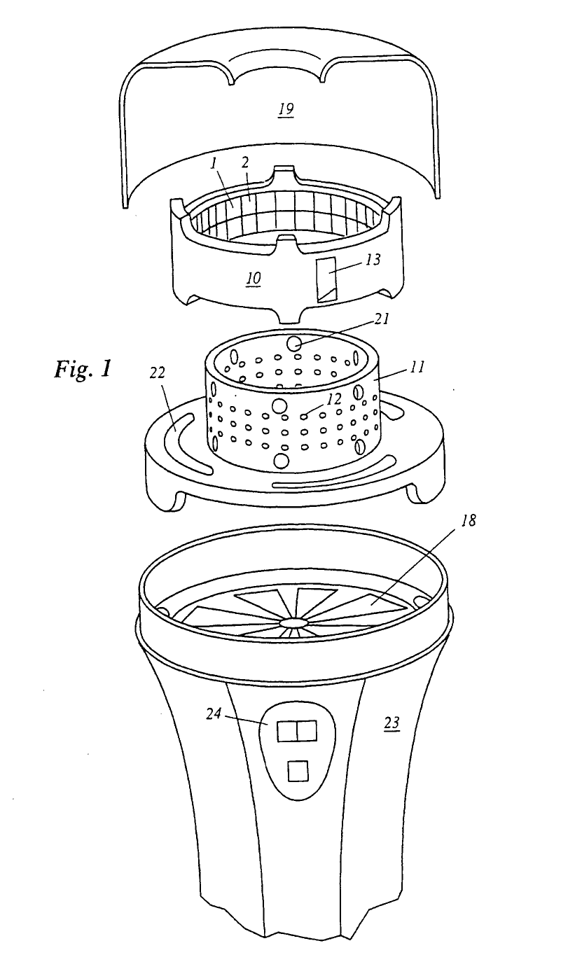

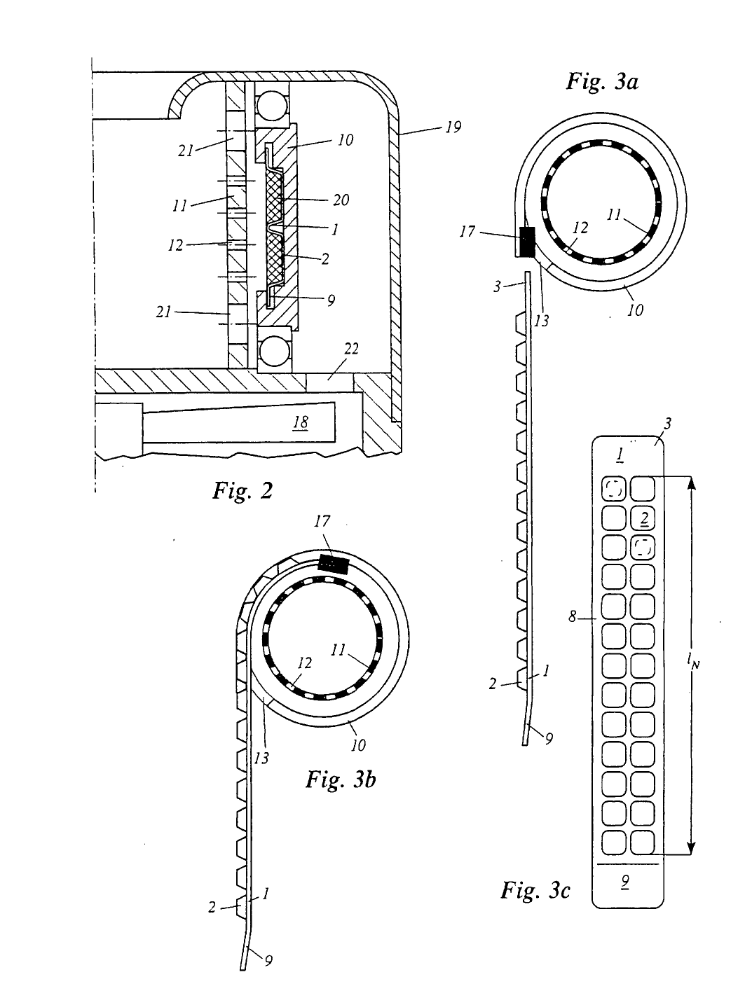

The exploded drawing shown in FIG. 1 illustrates the functional principle of an air germ collector according to the invention, which principle consists in the relative movement between collection medium 20 or accommodation 10 and screen cover 11. For a better understanding, the half-sided cross-sectional representation from FIG. 2 can be referred to; it corresponds to the embodiment shown in FIG. 1, to a great extent.

Accommodation 10 with collection strip 1 secured in the circumference direction can rotate and screen cover 11 is fixed in place. Likewise, screen cover 11 can be structured so that it can rotate, particularly if accommodation 10 is fixed in place. Also, it is possible to move collection strip 1 in the accommodation, in order to obtain the required relative movement. Collection strip 1, which is secured in the circumferential direction, is only indicated in FIG. 1. It can essentially be structured like strip 1 described below in connection with FIG. 3c.

Air is transported (drawn in) by fan 18 (disposed coaxial to the accommodation 10), in the following manner: Through the central opening of cover hood 19 and gas passage openings 12, the air reaches the surface of the collection medium, preferably nutrient medium 20 in depressions 2 of collection strip 1, where the inertial deposition of the airborne germs takes place (similar to the case of a conventional screen plate described initially).

In the case of highly germ-charged air, part of the air can flow through bypass bores 21 that can be closed. The closure mechanism (not shown) can be structured as a rotating ring that is concentric with screen cover 11 and rests against it, which ring has bores that are in complete or partial coverage with bypass bores 21, or not at all in coverage with them.

The air continues to flow past accommodation 10 (for example through gaps or bores), into the outer drum space, and is drawn through oblong holes 22 by fan 18, and ejected at the lower end of the air germ collector (not shown). The transport amount can be varied by changing the speed of rotation of the fan.

The control elements required for operation (not shown) are accommodated in housing 23 of the device, which is structured to be portable, along with a drive and power supply (network connector or battery). The operating elements and displays required for operation are disposed in an operating field 24. It is advantageous if the device is equipped in such a manner that it can detect the data that characterize the collection process (e.g. air throughput or fan speed of rotation, bypass setting, movement characteristics, such as angle of twist between accommodation 10 and screen cover 11), and store them in memory and/or transmit them to an external receiver, preferably in wireless manner.

Intake shaft 13 is provided for intake of collection strip 1, which is preferably performed in an automated manner, and is accessible by removing cover hood 19.

Alternatively, collection strip 1 can also be placed in manually. However, automated intake offers the advantage that the process of changing media is accelerated, simplified, and standardized. The risk of handling errors as well as contamination due to unintentional contact with medium 20 is reduced.

The movement of accommodation 10 relative to screen cover 11 can be brought about by way of a gear crown, by way of a magnetic coupling, or by way of other measures for transferring torque that are known to a person skilled in the art.

Because gas passage openings 12 travel a predetermined path over medium 20, the air stream does not always hit the same locations, and this reduces local drying out as well as the risk of depositing two germs at the same location.

The principle of the relative movement between screen cover 11 and nutrient medium or collection medium 20 can also be replicated, in advantageous manner, in the planar (not rotation-symmetrical) case, such as in the case of the conventional screen plate method. Also, arrangements of concentric drums (or other prismatic bodies) are possible, which perform an axial relative movement with regard to one another.

Collection strip 1 shown in FIG. 3c has depressions 2 filled with a collection medium, preferably a nutrient medium, as was already mentioned above. It is preferably produced from a plastic film that is stable in shape but flexible, by means of a deep-drawing method, but it can also be produced in some other manner.

In the region of strip 1 that leads during the automatic intake that is preferably provided, it has a broadened edge 3 as an engagement surface for friction-force grasping during intake into the air germ collector. Instead or in addition, other means can also be provided, which are adapted to engagement, particularly also shape-fit engagement, of intake means for automatically drawing the collection strip into an air germ collector. Possibilities are, for example, a crosswise slit, punched holes, an embossed crosswise groove, or lateral notches in edge 8 of strip 1.

For being held in accommodation 10, as well as for being guided while being drawn into the air germ collector, strip 1 possesses a lateral edge 8 that is not too narrow. In the case of the automatic intake that is preferably provided, this edge can be perforated to support the advance (for example by means of studded rollers or the like), similar to a film strip.

Strip 1 from FIG. 3c possesses a grab tab 9 (in its part that trails during intake), which bends away relative to the main plane of the strip, i.e. the imaginary connection plane of the edges of depressions 2, and which facilitates handling and furthermore can serve as a labeling surface. Suitable writing devices or labels can be used for labeling. Furthermore, however, labeling by means of a film structure that can be written on by means of a laser, an RFID (radio frequency identification device) arrangement (transponder chip), bar codes, matrix codes, etc., is also possible.

FIGS. 3a and 3b represent the intake process of a strip structured according to or similar to FIG. 3c into an air germ collector structured similar to FIG. 1.

As in FIGS. 1 and 2, a drum-shaped screen cover 11 having gas passage openings 12 is provided concentric to the drum-shaped strip accommodation 10. In operation, the germ-charged air flows from the drum interior through the gas passage openings 12 onto the surface of strip 1 in accommodation 10 that is drawn in at that time, and flows away farther to the side (completely drawn-in state not shown). Because of a relative movement of accommodation 10 and screen cover 11 with regard to one another (or movement of strip 1 in accommodation 10), the problems of local drying out and greater count deviations that were described initially in connection with screen plate methods rarely occur. It is particularly advantageous if this movement is performed in defined manner, so that each gas passage opening 12 moves along a predetermined path or angle range.

A relative movement performed in a defined manner is also particularly intended to mean a relative movement between screen cover 11 and collection strip 1 that is controlled over time, in such a manner that a predetermined relative path or angle movement takes place during a predetermined time of the collection process. In this connection, it can be particularly advantageous to perform a complete rotation or several complete rotations of accommodation 10 and screen cover 11 relative to one another (or a complete turn or several complete turns of strip 1 in accommodation 10) during the course of a collection process, so that each gas passage opening passes over an angle of 360 degrees or a whole-number multiple of it. If passage openings 12 are disposed in a repeating pattern, it is also advantageous to design the air germ collector so that it performs a relative movement between screen cover 11 and collection strip 1 during the predetermined time of the collection process, which movement corresponds to the distance or a whole-number multiple of the distance at which the pattern repeats.

An embodiment in which the predetermined relative path or angle movement during the predetermined time corresponds to the useful length l_{N} of the collection strip 1 provided with collection medium (cf. FIG. 3c) is also advantageous.

Particularly preferably, passage openings 12 on screen cover 11 are distributed in such a manner that each point of the surface of collection medium 20 comes into coverage with one of passage openings 12 at least once during the course of a collection process.

Passing over a predetermined path or angle in a controlled manner over time can be implemented by means of a mechanical control or a simple electrical control, but preferably, such a control is structured microelectronically. In this connection, it should also be possible to preselect different movement modes, for example when using different embodiments of screen covers 12 or collection strips 1.

Intake of the strip 1 essentially takes place tangentially through an intake shaft 13. Shaft 13 does not have to be elevated, as shown, but rather can also lie flush in the outside of accommodation 10.

Strip 1 is drawn in by an entrainment device 17 that runs tangentially in accommodation 10, or by a carriage guided in accommodation 10. The end position (not shown) is recognized by a stop or suitable sensors. In order to “seize” strip 1, entrainment device 17 has suitable manually or automatically operated means (not shown). These can be, for example, a clamping device or pins or catches, the latter of which engage into slits, holes, grooves, or notches, for example, of suitable strips 1 (all of them not shown in FIG. 3c).

Alternatively, strip 1 can be drawn in by means of endless conveyors. These can be rollers or rolls that transport the strip with friction force, but shape-fit transport of strip 1 is also possible, for example by means of studded rollers or gear wheels, if strip 1 has an edge perforation (as mentioned above) or a suitable ribbing. The endless conveyors can be disposed either only in the region of intake shaft 13, or also distributed over the circumference of accommodation 10.

A combination of endless conveyors in the region of intake shaft 13 with an entrainment device 17 can also be advantageous: Thus, for example, the intake can take place by way of endless conveyors until the leading strip end can be seized by the clamp, catch, etc. of entrainment device 17.

The intake can take place fully automatically or by means of manual control.

An automatic intake of collection strip 1 can advantageously be combined with the aforementioned embodiment variant, in such a manner that the relative movement between collection strip 1 and screen cover 11 during the collection process is obtained by movement of collection strip 1 in the accommodation. Preferably, a common drive can then be provided for the strip intake and the production of the relative movement in collection operation. The advantages that result from this are a reduction in the proportion of moving parts and a seal that might be implemented much more simply, under some circumstances.

Accordingly, while only a few embodiments of the present invention have been shown and described, it is obvious that many changes and modifications may be made thereunto without departing from the spirit and scope of the invention.

What is claimed is:

1. An air germ collector, comprising:
an accommodation;
a collection medium disposed in the accommodation;
acceleration means for accelerating air charged with germs, in order to allow inertial deposition of the germs from the air onto the collection medium; and
a cover covering the collection medium, said cover being provided with passage openings for the accelerated air,
wherein the cover and the collection medium are movable relative to one another, in a defined manner, during inertial deposition of the germs.

2. An air germ collector according to claim 1, wherein the air germ collector is designed such that the relative movement between the cover and collection medium is controlled over time, so that a predetermined relative path and/or angle movement takes place during a predetermined time of the collection process.

3. An air germ collector according to claim 2, wherein the collection medium is contained in a collection strip interchangeably held in the accommodation, and the predetermined relative path or angle movement during the predetermined time corresponds to a useful length (l_{N}) of the collection strip.

4. An air germ collector according to claim 2, wherein the passage openings are disposed in a repeating pattern, and wherein the relative movement between the cover and collection medium corresponds to a distance or a whole-number multiple of the distance at which the pattern repeats.

5. An air germ collector according to claim 1, wherein the accommodation is drum-shaped, and the cover is structured as a perforated drum disposed coaxial to the accommodation, in a drum interior surrounded by the accommodation.

6. An air germ collector according to claim 5, wherein the passage openings are uniformly distributed over the circumference of a drum mantle.

7. An air germ collector according to claim 5, wherein the accommodation is rotatable.

8. An air germ collector according to claim 7, wherein the perforated drum is fixed in place.

9. An air germ collector according to claim 5, wherein the acceleration means comprise a fan disposed coaxial with the accommodation.

10. An air germ collector according to claim 9, wherein the fan is disposed outside of the drum interior.

11. An air germ collector according to claim 1, wherein the collection medium is contained in a collection strip interchangeably held in the accommodation.

12. An air germ collector according to claim 11, wherein the collection strip is movable relative to the accommodation.

13. An air germ collector according to claim 12, further comprising intake means for drawing the collection strip into the accommodation in an automated manner.

14. An air germ collector according to claim 13, further comprising a common drive for the intake means and for moving the collection strip relative to the accommodation, during collection.

15. An air germ collector according to claim 1, wherein the passage openings are round.

16. An air germ collector according to claim 1, wherein the passage openings are uniform in shape, relative to one another.

17. An air germ collector according to claim 16, wherein a distance between adjacent passage openings, relative to one another, measured from an edge of each of the passage openings, is greater than a diameter of a circle inscribed in a passage opening.

18. An air germ collector according to claim 16, wherein a distance between adjacent passage openings, relative to one another, measured from an edge of each of the passage openings, is less than five times a diameter of a circle circumscribed around a passage opening.

19. An air germ collector according to claim 1, wherein the passage openings each have a minimum area of 0.2 mm².

20. An air germ collector according to claim 1, wherein the passage openings each have a maximum area of 1 mm².

21. An air germ collector according to claim 1, wherein the cover has bypass openings and wherein an area of a bypass opening is greater than an area of a passage opening.

22. An air germ collector according to claim 21, wherein the bypass openings can be closed.

23. An air germ collector according to claim 1, wherein the air germ collector is configured as a portable device.

24. An air germ collector according to claim 1, further comprising means for detecting data that characterize the collection process.

25. An air germ collector according to claim 24, wherein said means for detecting comprise means for wireless transmission of said data to an external receiver.
